(19) United States
(12) Patent Application Publication
(10) Pub. No.: US 2024/0084356 A1
(43) Pub. Date: Mar. 14, 2024

(54) RIBOSOMAL RNA SCAFFOLDS FOR PROTEIN-FREE AND TEMPLATE-FREE SYNTHESIS OF PEPTIDES

(71) Applicant: UNIVERSITY OF HOUSTON SYSTEM, Houston, TX (US)

(72) Inventors: Yuhong Wang, Houston, TX (US); Doris Xu, Houston, TX (US)

(73) Assignee: UNIVERSITY OF HOUSTON SYSTEM, Houston, TX (US)

(21) Appl. No.: 18/272,535

(22) PCT Filed: Dec. 22, 2021
(86) PCT No.: PCT/US2021/064971

§ 371 Date: Jul. 14, 2023

Related U.S. Application Data
(60) Provisional application No. 63/140,812, filed on Jan. 23, 2021.

(51) Int. Cl. C12P 21/02; C12N 9/00; C12N 9/10; C12N 15/113

(52) U.S. Cl. CPC C12P 21/02; C12N 9/104; C12N 9/93; C12N 15/113; C12Y 203/02012; C12Y 601/01; C12N 2310/12

(57) ABSTRACT

Embodiments of the present disclosure pertain to methods of synthesizing a peptide by associating at least one isolated ribosomal RNA (rRNA) fragment with a plurality of transfer RNA fragments linked to amino acids (tRNA fragments). The at least one rRNA fragment includes at least one domain that associates with the tRNA fragments. The at least one rRNA fragment catalyzes the synthesis of the peptide through peptidyl transfer of the amino acids from the tRNA fragments independently of messenger RNA (mRNA) templates. Additionally, the peptidyl transfer occurs independently of proteins. Further embodiments of the present disclosure pertain to systems for synthesizing a peptide. Such systems include the rRNA fragments of the present disclosure and optionally a plurality of tRNA fragments.

### CROSS-REFERENCE TO RELATED APPLICATIONS

The present application claims the benefit of U.S. Provisional Patent Application No. 63/140,812, filed on Jan. 23, 2021. The entirety of the aforementioned application is incorporated herein by reference.

### STATEMENT REGARDING FEDERALLY SPONSORED RESEARCH

This invention was made with government support under Grant No. R01 GM111452 awarded by the National Institutes of Health. The government has certain rights in the invention.

### BACKGROUND

Current methods of making peptides with non-natural amino acids have numerous limitations. Various embodiments of the present disclosure address the aforementioned limitations.

### SUMMARY

In some embodiments, the present disclosure pertains to methods of synthesizing a peptide by associating at least one isolated ribosomal RNA (rRNA) fragment with a plurality of transfer RNA fragments linked to amino acids (tRNA fragments). In some embodiments, the at least one rRNA fragment includes at least one domain that associates with the tRNA fragments. In some embodiments, the at least one rRNA fragment catalyzes the synthesis of the peptide through peptidyl transfer of the amino acids from the tRNA fragments independently of messenger RNA (mRNA) templates. In some embodiments, the peptidyl transfer occurs independently of proteins.

Additional embodiments of the present disclosure pertain to systems for synthesizing peptides. In some embodiments, the systems of the present disclosure include the rRNA fragments of the present disclosure. In additional embodiments, the systems of the present disclosure also include a plurality of tRNA fragments linked to amino acids. In some embodiments, the systems of the present disclosure also include at least one dimerization agent to promote dimerization of the at least one rRNA fragment.

Further embodiments of the present disclosure pertain to methods of preparing tRNA fragments linked to amino acids. In some embodiments, the methods of the present disclosure include associating transfer RNA fragments with amino acids in the presence of a charging agent that facilitates the linking of amino acids to the transfer RNA fragments. In some embodiments, the charging agent is a Yadb protein or a derivative thereof.

### BRIEF DESCRIPTION OF THE DRAWINGS

FIGS. 1A and 1B illustrate short rRNA fragments at the peptidyl transfer with a 2-fold rotated symmetry that are outlined red (ptc1b) and green (ptc1a). FIG. 1A shows the primary and tertiary structures from 4wpo. FIG. 1B shows the secondary structure. In ptc1a piece (green), two stem loops are truncated and replaced with short oligos from ptc1b (red lines).

FIGS. 2A-D show fluorescence resonance energy transfer (FRET) efficiency histograms of tRNA (oligo)-tRNA (oligo) or rRNA-rRNA interactions. FIG. 2A shows FRET efficiency histogram of 5′- and aminoacyl labeled RNA-lys pieces. The relative Cy3/Cy5 positions are shown in the insert. FIG. 2B shows FRET efficiency histogram of 3′-labeled ptc1b. The relative Cy3/Cy5 positions are shown in the insert. FIGS. 2C-2D show FRET efficiency histograms of FIGS. 2A-2B in the presence of 1 mM chloramphenicol, respectively.

FIGS. 3A1-B show high resolution MS of poly-lysine of N14,N14-(blue trace) and N15,N15-(green trace) labeled lysine monomers. Shifting of 18 suggests 9 lysine residues. FIGS. 3A1-A5 show mass spectra of lysine monomers and oligos. FIG. 3B shows tandem MS/MS on 2273 and 2291 peaks.

FIGS. 4A-4D show agarose native gel (stained with SybrTMGold) and FRET measurement of RNA dimers. FIG. 4A shows an agarose native gel with the following: Lane 1: single strand RNA ladder; Lane 2 and 3: PTC1b dimer; Lane 4: PTC1a dimer; and Lane 5: PTC1a/b dimer. FIG. 4B shows efficiency histograms between the RNA. The green and red starts showed the Cy3 and Cy5 labelling positions, respectively. FIGS. 4C-D show similar FRET efficiency histograms were observed with 5 and 15 mM Mg²⁺.

FIGS. 5A-E show FRET efficiency histograms between tRNA-like oligos at 10 mM of Mg²⁺. FIG. 5A shows the relative position of Cy3 (green star)/Cy5 (red star) labelled oligos. FIG. 5B shows a 30% denature PAGE Gel assay of Cy5 labelled oligos. Lane 1: CA_DNA; Lane 2: ACCCACCA; Lane 3: Lys-tRNALys_T1. FIGS. 5C-E show FRET of uncharged CA_DNA; charged Lys-tRNALys_T1; and Lysine-charged minihelix.

FIGS. 6A-C show gel shifting and MS to confirmation charging of non-natural amino acids. FIG. 6A shows a 30% denature PAGE of Cy5-labeled CCA (8 nt)-charged D-Cysteine (lane 2) and D-lysine (lane 3). FIG. 6B shows a 20% denature PAGE of Cy3 labeled D-lysine charged tRNA^{Lys} (lane 4), and its RNaseT1 digestion (Lane 2). The long tRNA bands in lane 4 is missing in lane 2 due to RNassT1 reaction. Lane one is the same marker as in Figure (A). Lane 3 is bromophenol blue and xylene cyanol. FIG. 6C is a mass spectrometry to show the molecular weight of CCA (8 nt)-D-lysine. Calculated MW is 2582. Observed MW is 2584.

FIG. 7 is a Rapi-flex mass spectrum of CACCA-Phe (brown trace). Incubating this oligo with the ribozyme (1 microM blue trace; 2.5 microM pink trace) in the presence of Mg²⁺ generated a peak around 1975, which is consistent with a tripeptide CACCA-(Phe)₃. Leaving out of Mg²⁺ abolished the formation of this peak, which is a control experiment.

FIGS. 8A-B show smFRET to show substrate proximity between amino acid charged oligos that are labeled with FRET-paired dyes. FIG. 8A is a FRET between Cy3-L-Lysine-CCA (8 nt), and Cy5-D-Lysine-CCA (8 nt). FIG. 8B is a FRET between Cy3-D-Lysine-CCA (8 nt), and Cy5-D-Lysine-CCA (8 nt).

### DETAILED DESCRIPTION

It is to be understood that both the foregoing general description and the following detailed description are illustrative and explanatory, and are not restrictive of the subject matter, as claimed. In this application, the use of the singular includes the plural, the word “a” or “an” means “at least one”, and the use of “or” means “and/or”, unless specifically stated otherwise. Furthermore, the use of the term “including”, as well as other forms, such as “includes” and “included”, is not limiting. Also, terms such as “element” or “component” encompass both elements or components comprising one unit and elements or components that include more than one unit unless specifically stated otherwise.

The section headings used herein are for organizational purposes and are not to be construed as limiting the subject matter described. All documents, or portions of documents, cited in this application, including, but not limited to, patents, patent applications, articles, books, and treatises, are hereby expressly incorporated herein by reference in their entirety for any purpose. In the event that one or more of the incorporated literature and similar materials define a term in a manner that contradicts the definition of that term in this application, this application controls.

Non-natural amino acids are resistant to cellular proteases. Therefore, peptides with non-natural amino acids have numerous applications. For instance, peptides with non-natural amino acids are ideal drug candidates that can be delivered into cells without degradation. However, due to the remarkable fidelity of ribosomal protein synthesis, the synthesis of peptides with non-natural amino acids remains challenging. Numerous embodiments of the present disclosure address the aforementioned limitations.

In some embodiments, the present disclosure pertains to methods of synthesizing a peptide. In some embodiments, the methods of the present disclosure include associating at least one ribosomal RNA (rRNA) fragment with a plurality of transfer RNA fragments linked to amino acids (tRNA fragments). In some embodiments, the at least one rRNA fragment includes at least one domain that associates with the tRNA fragments. Thereafter, the at least one rRNA fragment catalyzes the synthesis of the peptide through peptidyl transfer of the amino acids from the tRNA fragments. In some embodiments, the peptidyl transfer occurs independently of proteins, such as peptidyl transferases. In some embodiments, the peptidyl transfer occurs independently of messenger RNA (mRNA) templates.

Additional embodiments of the present disclosure pertain to systems for synthesizing a peptide. In some embodiments, the systems of the present disclosure include at least one rRNA fragment with at least one domain capable of associating with tRNA fragments linked to amino acids. In some embodiments, the at least one rRNA fragment catalyzes the synthesis of peptides through peptidyl transfer of the amino acids from the tRNA fragments. In some embodiments, the systems of the present disclosure also include a plurality of tRNA fragments. In some embodiments, the systems of the present disclosure also include a dimerization agent for dimerizing the at least one rRNA fragment.

As set forth in more detail herein, the methods and systems of the present disclosure can have numerous embodiments. For instance, the methods and systems of the present disclosure can utilize numerous rRNA fragments that are capable of associating with numerous tRNA fragments in order to synthesize numerous types of peptides.

Ribosomal RNA Fragments

The systems and methods of the present disclosure can include numerous types of rRNA fragments. For instance, in some embodiments, the rRNA fragments are in dimerized form. In some embodiments, the rRNA fragments are in homodimer form. In some embodiments, the rRNA fragments are in heterodimer form.

In some embodiments, the rRNA fragments are in isolated form. For instance, in some embodiments, the rRNA fragments are isolated from their native environments, such a cellular environments. In some embodiments, the rRNA fragments are isolated from cellular materials, such as proteins. In some embodiments, the rRNA fragments are in synthetic form.

The rRNA fragments of the present disclosure can include various domains. For instance, in some embodiments, the rRNA fragment includes a ribosomal peptidyl transferase center (PTC).

The rRNA fragments of the present disclosure can have various lengths. For instance, in some embodiments, the rRNA fragments have lengths of less than 180 nucleotides. In some embodiments, the rRNA fragments have lengths of less than 150 nucleotides. In some embodiments, the rRNA fragments have lengths of less than 125 nucleotides. In some embodiments, the rRNA fragments have lengths of less than 110 nucleotides.

The rRNA fragments of the present disclosure can also include numerous different fragments of rRNA. For instance, in some embodiments, the rRNA fragments include, without limitation, a first rRNA fragment, a second rRNA fragment, or combinations thereof. In some embodiments, the rRNA fragments include a first rRNA fragment and a second rRNA fragment. In some embodiments, the first rRNA fragment and the second rRNA fragment are in the form of a heterodimer. In some embodiments, the first rRNA fragment includes the sequence AUGUCGGCUCGUCGCAUCCUGGGACUGAAGAAGGUCCCAAGGGUUGGGCUGUUC GCCCAUUAAAGCGGCACGCGAGCUGGGUUCAGAACGUCGUGAGACAGUUCGGUC (SEQ ID NO: 1), a DNA variation thereof, or a derivative thereof. In some embodiments, the first rRNA fragment includes a DNA variation of SEQ ID NO: 1.

In some embodiments, the first rRNA fragment includes a derivative of SEQ ID NO: 1. In some embodiments, the derivative shares at least 90% sequence identity with SEQ ID NO: 1. In some embodiments, the derivative shares at least 80% sequence identity with SEQ ID NO: 1. In some embodiments, the derivative shares at least 70% sequence identity with SEQ ID NO: 1. In some embodiments, the derivative shares at least 60% sequence identity with SEQ ID NO: 1. In some embodiments, the derivative is a 50-150 nucleotide length derivative of SEQ ID NO:1.

In some embodiments, SEQ ID NO: 1 (or a derivative thereof) forms hydrogen bonds with a helix structure (e.g., helix of a tRNA fragment) at or near the G51 position, the G52 position, or combinations thereof. Additionally, SEQ ID NO: 1 (or a derivative thereof) can facilitate peptidyl transfer at various positions. For instance, in some embodiments, peptidyl transfer occurs at or near the U4 position, the G51 position, the G75 position, the A79 position, the A81 position, the U83 position, the A100 position, or combinations thereof.

In some embodiments, the second rRNA fragment includes the sequence AGACCCCGUGGAGCUUUACUGCAGCCGAAGAAGGCGGGCAGUUUGACUGGGGCG GUC AA AAAAGUUACCCC GGGGA UAACAGGCUGAUCUCGUGAGGAG GUUUGGCACCUC (SEQ ID NO: 2), a DNA variation thereof, or a derivative thereof. In some embodiments, the second rRNA fragment includes a DNA variation of SEQ ID NO: 2. For instance, in some embodiments, the DNA variation of SEQ ID NO: 2 includes the DNA sequence ATTCA or a derivative thereof.

In some embodiments, the second rRNA fragment includes a derivative of SEQ ID NO: 2. In some embodiments, the derivative shares at least 90% sequence identity with SEQ ID NO: 2. In some embodiments, the derivative shares at least 80% sequence identity with SEQ ID NO: 2. In some embodiments, the derivative shares at least 70% sequence identity with SEQ ID NO: 2. In some embodiments, the derivative shares at least 60% sequence identity with SEQ ID NO: 2. In some embodiments, the derivative is a 50-150 nucleotide length derivative of SEQ ID NO: 2.

In some embodiments, the rRNA fragments of the present disclosure include both SEQ ID NO: 1 and SEQ ID NO: 2. In some embodiments, the rRNA fragments of the present disclosure include a heterodimer of SEQ ID NO: 1 and SEQ ID NO: 2.

Dimerization Agents

In some embodiments, the systems of the present disclosure also include dimerization agents to promote the dimerization of the rRNA fragments of the present disclosure. In some embodiments, the dimerization agents include a metal, such as Mg⁺². In some embodiments, the dimerization agents include MgCl₂.

Association of rRNA Fragments with tRNA Fragments

Various methods may be utilized to associate rRNA fragments with tRNA fragments. For instance, in some embodiments, the association occurs by mixing the rRNA fragments with the tRNA fragments. In some embodiments, the association occurs by heating. In some embodiments, the association occurs in solution. In some embodiments, the association occurs in vitro. In some embodiments, the association occurs independently of any mRNA templates. In some embodiments, the association occurs in the absence of any catalytic proteins.

Transfer RNA Fragments

The methods and systems of the present disclosure can utilize various types of tRNA fragments. In some embodiments, tRNA fragments include tRNA fragments that are linked to amino acids. In some embodiments, the amino acids are linked to tRNA fragments through ester bonds between carboxyl groups of the amino acids and 3′-OH groups of the tRNA fragments.

In some embodiments, the tRNA fragments include one or more mini-helices. In some embodiments, the one or more mini-helices are defined by one or more loops of nucleotides. In some embodiments, the tRNA fragments include at least one mini-helix. In some embodiments, the tRNA fragments include at least two mini-helices. In some embodiments, the tRNA fragments include at least three mini-helices.

In some embodiments, the tRNA fragments are in the form of short RNAs (sRNAs). In some embodiments, the sRNAs have lengths of less than about 50 nucleotides. In some embodiments, the sRNAs have lengths of less than about 20 nucleotides. In some embodiments, the sRNAs have lengths of less than about 10 nucleotides. In some embodiments, the sRNAs have lengths of less than about 9 nucleotides. In some embodiments, the sRNAs have lengths of about 5 to 8 nucleotides.

Preparation of tRNA Fragments

In some embodiments, the methods of the present disclosure also include a step of preparing the amino-acid containing tRNA fragments of the present disclosure. In some embodiments, the preparation is facilitated by a charging agent that facilitates the linking of amino acids to tRNA fragments. In some embodiments, the charging agent is a Yadb protein or a derivative thereof.

Additional embodiments of the present disclosure pertain to methods of preparing transfer RNA fragments linked to amino acids by associating transfer RNA fragments with amino acids in the presence of a charging agent such that the charging agent facilitates the linking of amino acids to the transfer RNA fragments. In some embodiments, the charging agent is a Yadb protein or a derivative thereof. In some embodiments, the amino acids include, without limitation, canonical amino acids, non-canonical amino acids, non-natural amino acids, and combinations thereof.

In some embodiments, the Yadb protein includes a derivative of the Yadb protein. In some embodiments, the derivative of the Yadb protein includes mutations that optimize the binding of the Yadb protein to the tRNA fragments.

In some embodiments, the Yadb protein is defined by SEQ ID NO: 3. In some embodiments, the Yadb protein or its derivative is defined by a sequence that shares at least 60% sequence identity with SEQ ID NO: 3. In some embodiments, the Yadb protein or its derivative is defined by a sequence that shares at least 70% sequence identity with SEQ ID NO: 3. In some embodiments, the Yadb protein or its derivative is defined by a sequence that shares at least 80% sequence identity with SEQ ID NO: 3. In some embodiments, the Yadb protein or its derivative is defined by a sequence that shares at least 90% sequence identity with SEQ ID NO: 3. In some embodiments, the Yadb protein or its derivative is defined by a sequence that shares at least 95% sequence identity with SEQ ID NO: 3.

In some embodiments, the Yadb protein includes a derivative of SEQ ID NO: 3 that includes mutations that enhance its binding to tRNA fragments. For instance, in some embodiments, the at least one mutation includes at least one mutation in one or more of the following residues: Glu 159, Leu 156, Ile 114, Phe 161, Tyr 115, Tyr 172, or combinations thereof. In some embodiments, Glu 159 is mutated to lysine. In some embodiments, Ile 114 is mutated to glycine.

Peptidyl Transfer of Amino Acids to Form Peptides

The methods and systems of the present disclosure may be utilized to peptidyl transfer various types of amino acids from tRNA fragments and thereby form various types of peptides. For instance, in some embodiments, the amino acids include canonical amino acids, non-canonical amino acids, non-natural amino acids, and combinations thereof.

In some embodiments, the amino acids include non-canonical amino acids. In some embodiments, the amino acids include canonical amino acids. In some embodiments, the amino acids include non-natural amino acids. In some embodiments, the non-natural amino acids include D-forms of natural amino acids (e.g., D-cysteine and and/or D-Lysine).

In some embodiments, the peptidyl transfer occurs randomly without proofreading. In some embodiments, the peptidyl transfer occurs without interaction with anti-codon domains of tRNA fragments. In some embodiments, the peptidyl transfer occurs independently of mRNAs.

In some embodiments, the formed peptides include peptides with lengths ranging from about 2 amino acids to 100 amino acids. In some embodiments, the peptides include lengths ranging from about 2 amino acids to 50 amino acids. In some embodiments, the peptides include lengths ranging from about 2 amino acids to 10 amino acids. In some embodiments, the peptides include canonical amino acids, non-canonical amino acids, non-natural amino acids, or combinations thereof. In some embodiments, the peptides include non-canonical amino acids, non-natural amino acids, or combinations thereof.

Applications and Advantages

The methods and systems of the present disclosure can provide numerous applications and advantages. For instance, in some embodiments, the methods and systems of the present disclosure can be utilized to synthesize any natural or non-natural peptides without the need for any enzymes or chemicals other than a dimerization agent (e.g., MgCl₂). Moreover, in some embodiments, the systems and methods of the present disclosure can be utilized to synthesize peptides in a template-free and proofreading-free manner.

As such, in some embodiments, the systems and methods of the present disclosure can be utilized to synthesize drug-lead peptides in a facile and random manner. In some embodiments, the systems and methods of the present disclosure can be utilized to generate large amino acid-oligo libraries without restraint on amino acid species or chirality. In some embodiments, the systems and methods of the present disclosure can be utilized to synthesize mixtures of peptides that include both natural and non-natural peptides. In some embodiments, the synthesized peptides can be used to screen drug candidates and other functional peptides.

### Additional Embodiments

Reference will now be made to more specific embodiments of the present disclosure and experimental results that provide support for such embodiments. However, Applicant notes that the disclosure below is for illustrative purposes only and is not intended to limit the scope of the claimed subject matter in any way.

### Example 1. Protein-Free Ribosomal RNA Scaffolds can Assemble Poly-Lysine Oligos from Charged tRNA Fragments

Ribosomal protein synthesis is a central process of the modern biological world. Because the ribosome contains proteins itself, it is very important to understand its precursor and evolution. Small ribozymes have demonstrated the principle of “RNA world” hypothesis, but protein free peptide ligase remains elusive.

In this Example, Applicant has identified two fragments in the peptidyl transfer center that can synthesize a 9-mer poly-lysine in a solution that contains Mg²⁺. This result is deduced from isotope-shifting in high resolution MS. To Applicant's knowledge, this is the longest peptide oligo that can be synthesized by a pure ribozyme.

Via single molecule fluorescence resonance energy transfer (FRET) experiments, Applicant has demonstrated in this Example that the ligase mechanism was likely by substrate proximity via dimerization. As such, Applicant envisions that these RNA fragments can be useful to synthesize template free natural and non-natural peptides while serving as model systems for peptidyl transfer reaction mechanisms and shedding light to the evolution of ribosome.

### Example 1.1. Background

The ribosome is the universal molecular device that synthesizes all proteins in cells. These proteins are responsible for executing every major cellular function in the modern biological world. Ribosomes have two subunits (30S and 50S), each containing 20-50 proteins and 1-3 RNA polymers. Since modern ribosomes are made of the proteins that they synthesize, there must have existed protein-free ancestors that exhibited peptide ligase activity.

The origin of peptide ligase is one of the most fundamental evolution questions to understand the transition from the “ancient RNA-” to the “modern protein-” worlds. Before the divergence of the three life domains, the common ancestor of all organisms, LUCA (last universal common ancestor), contained nearly complete components for protein translation, implying that the development of the ribosome is an early event in the history of evolution. The lack of primitive intermediates also makes the origin of the ribosome elusive.

Structural and biochemical assays have indicated that the peptidyl transfer center of the ribosome is the most ancient component and contains no protein. However, the search for an RNA-only peptide ligase is still unsuccessful. Nevertheless, very small non-rRNA ribozymes prepared through in vitro selection can catalyze peptide bond between short aminoacylated RNA oligos. In addition, aminoacyl minihelix, which is more similar to current tRNA substrates, can form peptide bond with puromycin-containing oligos that complemented to the CCA sequence of the minihelix.

These results demonstrated the feasibility of RNA-only peptide ligase. However, truncated and in vitro selected constructs of longer RNA chains from the domain V of the large subunit rRNA 23S, in which the peptidyl transfer center resides, were inactive of peptide bond formation. In the center of domain V, approximately 180 nucleotides formed a two-fold rotation symmetry where each half forms H-bond with the A- and P-site tRNAs, respectively. These two fragments formed similar secondary and tertiary structures with little sequence homologs. Furthermore, residues G2553 G2252/G2253 (bacterial numbering) in the A- and P-loops formed H-bonds with the tRNAs to position them for optimized reaction orientation.

### Example 1.2. Experimental Design

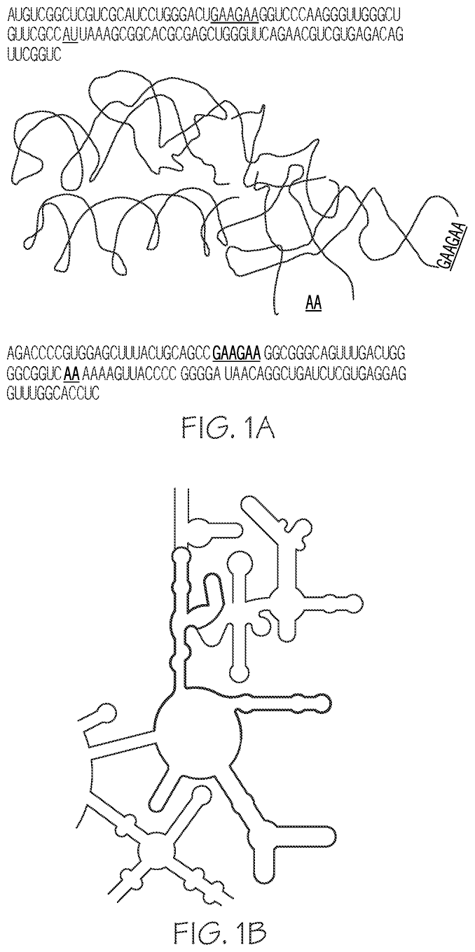

Although proximity is not sufficient for peptide bond formation, it is a precondition. Thus, Applicant searched for the protein-free ligase using single molecule FRET detection. By attaching rRNA chains to a glass surface under a microscope, proximal binding of tRNAs is easy to detect without ambiguity. Surprisingly, Applicant found that a 108 nt rRNA scaffold (residues 2503-2610, named ptc1b and outlined red in FIGS. 1A-1B alone can bring two labeled tRNA 3′-fragments into very close distance, which is indicated by a FRET efficiency of 0.6.

Further FRET experiments on labeled ptc1b suggested that closeness of the tRNA fragments was due to rRNA dimerization, which depended on Mg²⁺. By superimposing this rRNA piece into its symmetric counterpart in domain V (pdb ID 4wpo), Applicant identified another rRNA scaffold (green outline in FIGS. 1A-1B, named ptc1a). The fragment contains 2060 to 2501 fragments, with two stem loops truncated and filled with short RNA oligos guided by the superposition. Both sequences are shown in FIGS. 1A-1B.

Next, Applicant tested the ligase activities of the homo/hetero rRNA dimers with charged Lysine-tRNAlysine after RNaseT digestion. Surprisingly, Applicant identified formation of a 9-mer poly-lysine. This result is corroborated by mass-shifting of product incorporating N15,N15-labeled lysine in high resolution Mass Spec. Applicant found that rRNA alone can synthesize not just single peptide bonds but multiple ones, suggesting co-appearance and co-evolution of peptides and RNAs in the primordial world.

The template-free protein synthesis seems to suggest a module-based evolution hypothesis, in which 3D structure interactions without sequence constrain drive the evolution. The RNA scaffolds also provided a model to study the chemical mechanism of the peptidyl transfer reactions, which remains a fundamental challenge. Additionally, Applicant envisions that this template-free, proofreading-free protein polymerase will be useful to synthesize non-natural peptides for bioengineering applications.

### Example 1.3. Preparation of RNA Scaffolds

The 5′-biotinylated ptc1b RNA and ptc1a RNA molecules are purchased from IDTDNA. The 3′-ends are labeled with the “3′ EndTag™ DNA End Labeling System” from the Vector@Laboratories. The Cy3/Cy5-maleimide dyes are purchased from GE Life Sciences. The labeling efficiency is approximately 50% based on spectrophotometer absorptions.

### Example 1.4. tRNA Charging and Rnase T1 Digestion

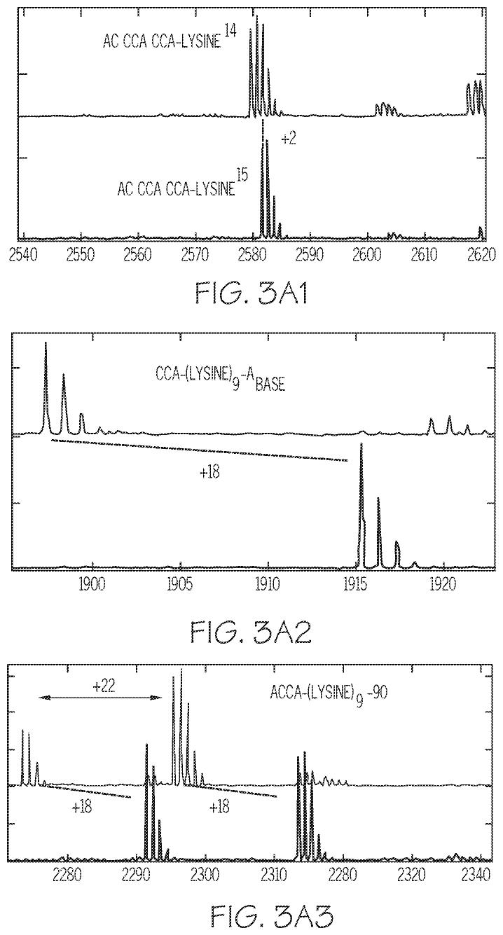

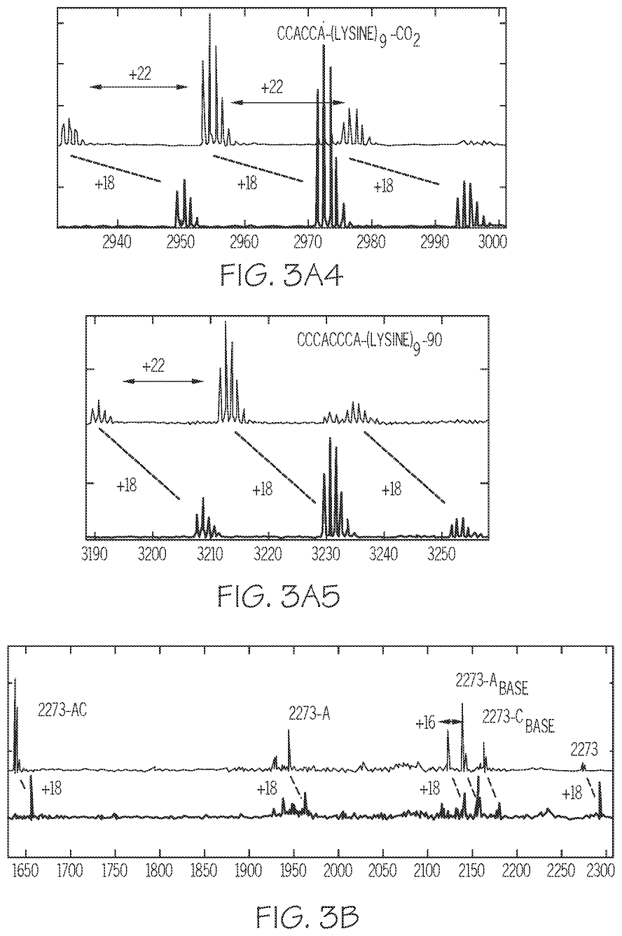

The tRNAlysine powder was purchased from chemical-block.com and was charged with normal-(Millipore-Sigma), C14-(PerkinElmer), or N15,N15-(Millipore-Sigma) labeled lysine as described previously. The charging efficiency is approximately 1000 pmol/A260 based on absorption and C14 radioactivity. Afterward, 4 A260 of charged tRNAlysine (theoretical amount 1800 pmol/A260) was digested with 1,000 units of RNase T1 (thermofisher) under denature condition following the manufacture's manual. The lysine piece was first phenol extracted and precipitated, and re-dissolved with water. Then it was enriched with the Monarch® RNA Cleanup kit. First, the RNA oligos (>25 nt) were captured by the Monarch® binding column by adding 1× volume of ethanol. Then another 1× volume of ethanol was added to the run-through liquid, and loaded onto a new filter to bind the smaller pieces (<25 nt). After washing, the RNA-lysine piece was eluted with 50 μl of RNase-free water. The expected RNA-lysine pieces were supported by high resolution MALDI MS (Mass Spectrometry facility, University of Alabama), and named “RNA-lys” (FIG. 3A1).

### Example 1.5. RNA-Lysine Labeling

The RNA-lysine piece was labeled in two positions: at the lysine moiety and the 5′-end of the RNA. For labeling at the lysine moiety, Cy3 or Cy5 NHS-dyes (GE Life Sciences) were dissolved in DMSO and added directly into the RNA-lysine solution mentioned above (final dye concentration ˜500 μM from 10 mM stocks). The labeling reaction was incubated at 37° C. for 2 hours, and the excess dye was removed via a G25 size-exclusive column. Then the Cy3- or Cy5-labeled RNA-lysine was precipitated and resuspended in 10-20 μl of RNase-free water. Labeling at the 5′-end was prepared with the “5′ EndTag™ DNA/RNA End Labeling System” from the Vector@Laboratories. The labeling efficiency of both positions are approximately (0.6 μM of dye)/(A260 of RNA).

### Example 1.6. Single Molecule FRET Experiments

The total internal reflection fluorescence microscope (TIRF) was based on a Nikon Eclipse Ti2-E inverted microscope with two auto-turrets and two CMOS cameras. The top turret reflects the laser for TIRF illumination, and the bottom turret split the FRET signals into two CMOS cameras based on wavelengths. For all measurements, sample concentrations are in the range of 10-100 nm. An oxygen scavenger cocktail (3 mg/mL glucose, 100 mg/mL glucose oxidase, 48 mg/mL catalase, and 2 mM trolox) was added to the channel before imaging to prevent photo bleaching.

### Example 1.7. Results and Discussion

First, Applicant has observed high FRET efficiency between Cy3/Cy5-labeled RNA-lys in the presence of ptc1b. The reason ptc1b was chosen, instead of the total 180 nt from the literature, was due to the complicated folding for longer RNAs. The sequence of ptc1b reproduced the same secondary structure as in the ribosome, while the 180 nt chain did not (Mfold Web Server).

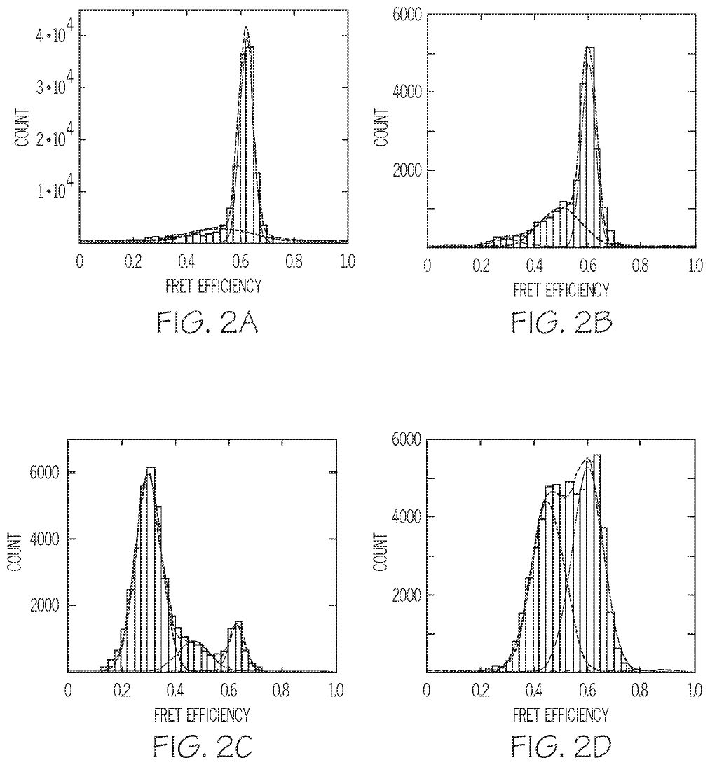

As shown in FIG. 2A, the Cy3 and Cy5 dyes are located at the aminoacyl and the 5′-terminal of RAN-lys, respectively. 1 μM of ptc1b was incubated with 0.5 μM of Cy3/Cy5-labeled RNA-lys in the presence of 0 or 15 mM MgCl₂. The mixture was incubated at 37° C. for 10 min and diluted with either water or 15 mM MgCl₂ solution at room temperature. This solution was loaded into the sample cell and attached to the surface via biotin-streptavidin interaction.

The FRET efficiencies are calculated as I acceptor/(Idonor+Iacceptor), in which Ixx is the fluorescence intensity in the donor or acceptor channel. In the presence of Mg²⁺, a FRET species centered at 0.62 was observed, which corresponds to 50 Å (R0=55 Å). From the X-ray structure (4wpo), the amino acid of A-site tRNA to the 5′- of ACCCACCA at P-site (tRNAlys sequence) is approximately 43 Å. This result suggested that the ptc1b can dimerize to mimic the two-fold symmetric peptidyl transfer center; and the tRNA fragments bind to the dimer in a similar way as tRNAs binding to the ribosome.

Second, the ptc1b/a dimerization hypothesis was tested. The ptc1b is labeled on the 3′-ends (C2610) with Cy3 and Cy5, respectively. Their dimerization is directly monitored with FRET. As predicted, FRET between Cy3-ptc1b and Cy5-ptc1b (FIGS. 2A-2B), exhibited a high FRET species centered at 0.60. Without Mg²⁺, this species disappeared, indicating that the dimerization also needs Mg²⁺. This FRET value corresponds to 51.4 Å, where the theoretical distance from the structure is about 23 Å (by aligning ptc1b to its hypothetical symmetric counterpart, insert of FIG. 2A). This discrepancy indicates a looser structure in the RNA-only dimers than in the well-packed single-chain peptidyl transfer center. Variation of linker length, quantum yields and orientation may contribute to the difference as well.

Next, Applicant synthesized ptc1a molecule, in which most of the sequence were from rRNA, but two long stem loop structures were replaced with short oligos, based on its alignment with ptc1b. The ptc1a piece was also labeled at the 3′-end, and hetero-dimer formation of ptc1a/b was detected. The FRET efficiencies are almost the same in the hetero dimer experiment. These high FRET species (ptc1b homo-dimer and the ptc1a/b hetero-dimer) are likely the reasons to bring the RNA-lys species together that generated the high FRET signals in FIG. 2A.

Third, the 0.62 FRET species between RNA-lys in FIG. 2A was diminished by chloramphenicol, but the 0.60 FRET species between ptc1b homodimer in FIG. 2B was not (FIGS. 2C-2D). Chloramphenicol is an antibiotic that binds to the same location as the aminoacyl moiety of A-site tRNA. If the prospected structures are correct in FIGS. 2A-2B, then adding chloramphenicol should interfere the high FRET species in FIG. 2A, but not in FIG. 2B. This is because chloramphenicol directly competes the binding pocket with RNA-lys, but does not compete with the ptc1a/b dimerization without the short tRNA fragments.

Applicant observed the aforementioned effect in FIGS. 2C-2D. The 0.62 FRET species diminished while a 0.3 FRET species emerged in the RNA-lys experiments, indicating inhibition of RNA-lys binding. Meanwhile, the 0.6 FRET species remained unchanged in the ptc1a/b dimerization experiments. Furthermore, the small peak at 0.5 FRET species in FIG. 2B is significantly larger when drug is present in FIG. 2D, which means chloramphenicol may have induced more dimers in a more loose structure. FIGS. 2C-2D suggest that the structural assignments of the FRET species in FIGS. 2A-2B are reasonable.

Fourth, Applicant has found that a 9-mer poly-lysine oligo is formed by the ptc dimers. In these experiments, 1 μM of ptc1b homo-dimer or ptc1a/b hetero-dimer was incubated with 15 mM MgCl₂ and 1 μM of RNA-lys, in which normal or N₁₅,N₁₅-labeled lysine was used. The mixtures were incubated at 37° C. for 1 hr or 3 hrs (no difference was detected in mass spectrometry analysis), run through a G25 desalting column, and analyzed with high resolution MALDI. Multiple mass peaks exhibited +18 shift in comparison of the N₁₄ to the N₁₅ labeled experiments, showing incorporation of 9 lysine residues (2 Da mass shift per residue).

Tandem MS/MS spectra were obtained on the 2273 peak and its +18 shifting peak in the normal and N₁₅, N₁₅-labeled experiments, respectively (FIG. 3B). These spectra showed consistent +18 shifting at multiple positions, indicating there were 9-mer lysine in the 2273/2291 peaks. Mass calculation indicated that these peaks correspond to CCA-K9 (FIG. 3A2), ACCA-K9 (FIG. 3A3), CCACCA-K9 (FIG. 3A4), and CCCACCA-K9 (FIG. 3A5).

For the MS/MS of 2273 peak, species losing A or C base on the riboses and losing complete A- and AC-nucleotides are observed. Although the starting material was confirmed to be ACCCACCA-K (FIG. 3A1), Applicant did not observe the product of 9-mer poly-lysine on the 8-nt fragment. Such an observation is probably due to the loss of the terminal A residue, either during the reaction incubation or MALDI measurements.

In summary, short rRNA fragments, containing the same sequences as those at the peptidyl transfer center, dimerized and brought tRNA fragments to proximity. High resolution mass spectrometry analysis indicated that these dimers have synthesized not one but eight peptide bonds to form a 9-mer RNA-K9 molecule out of the Lysine-charged tRNA fragment (ACCCACCA-K).

Poly-lysine has shown to adopt β-sheets structure, which intercalates well into the double helix grooves. The ancient molecules tRNA synthetases and ribosome reserved these interactions. The tRNA synthetases depend on their β-sheets structures to charging tRNAs, while some ribosomal proteins bind to rRNA's grooves. For example, L15 and S11 proteins demonstrated such interaction.

This relatively long oligo synthesis suggests that primitive peptide ligase may have higher capacity than previously envisioned. Therefore, a peptide/nucleic acid co-evolution route is possible, as suggested in the literature. Theoretic study has suggested that optimizing the interaction between RNA's double helix and peptide's β-sheet could drive the evolution of both species without sequencing fidelity. Indeed, diverse RNA sequences can form similar structural modules. The ptc1a and 1b molecules in this report are examples.

Applicant also proposes that the aforementioned peptide ligase can be a starting point in bioengineering to synthesize non-natural peptides without mRNA templates, which is difficult to incorporate by the native ribosome and is seriously pursued for drug development. Synthesis of a 9-mer peptide oligo is a promising starting point to engineer a more powerful peptide ligase to make longer peptide chains without overcoming the rigorous selection and proofreading steps of the native ribosome. Therefore, unnatural/mixed peptide chains can be synthesized in the future.

### Example 2. Additional Experimental Results

In this Example, Applicant provides additional experimental results related to the experimental results in Example 1.

### Example 2.1. Gel Confirmation of RNA Scaffolds Dimer

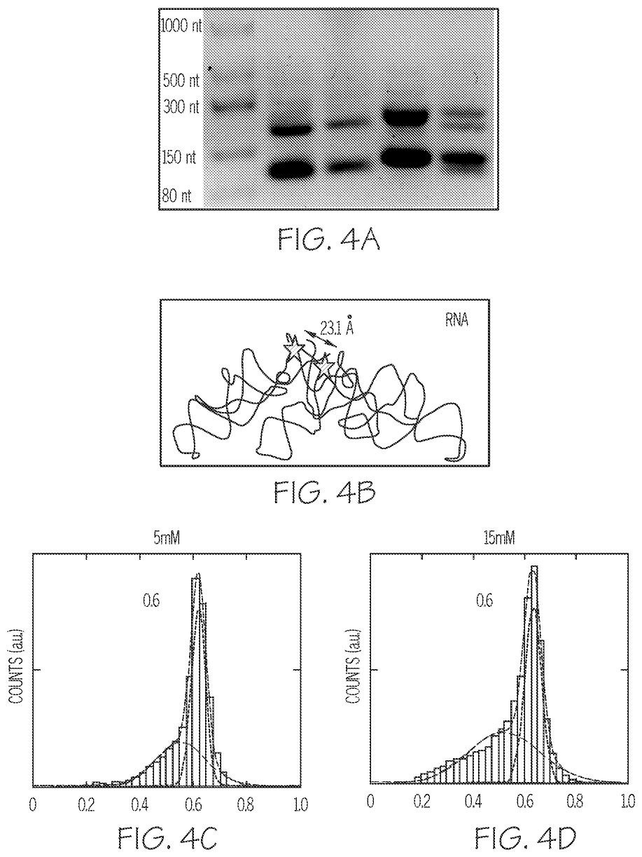

The dimerization of the RNA scaffolds is the main driving force and catalytical mechanism for the enzymatic synthesis of natural and non-natural peptides. In addition to smFRET data to show the dimer formation, Applicant has confirmed the dimer via non-denature agarose gel (FIGS. 4A-D). The dimers were indicated by the gel shifting positions between 150 nt-300 nt ladder, which were twice as much as the monomers around 100 nt.

### Example 2.2. Other tRNA-Like Short Oligos as Ribozyme Substrates

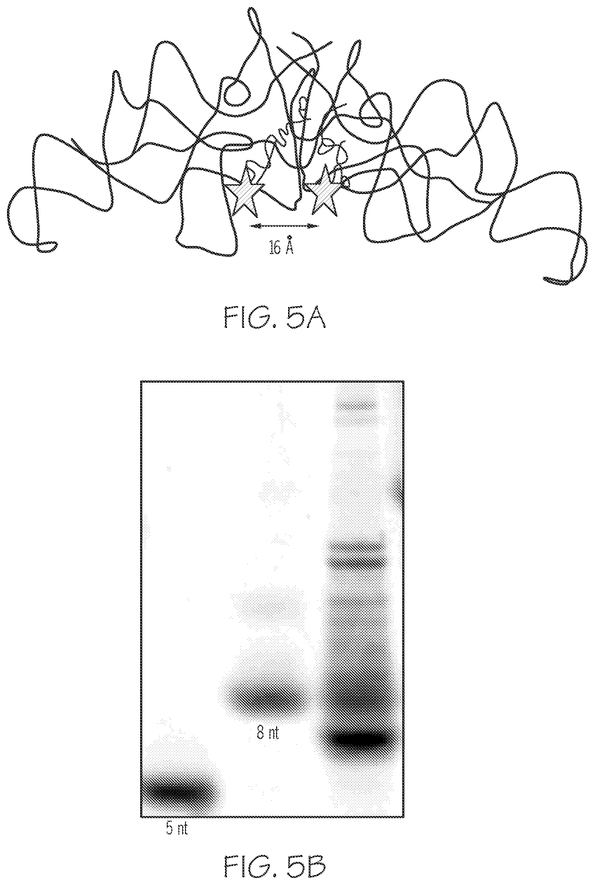

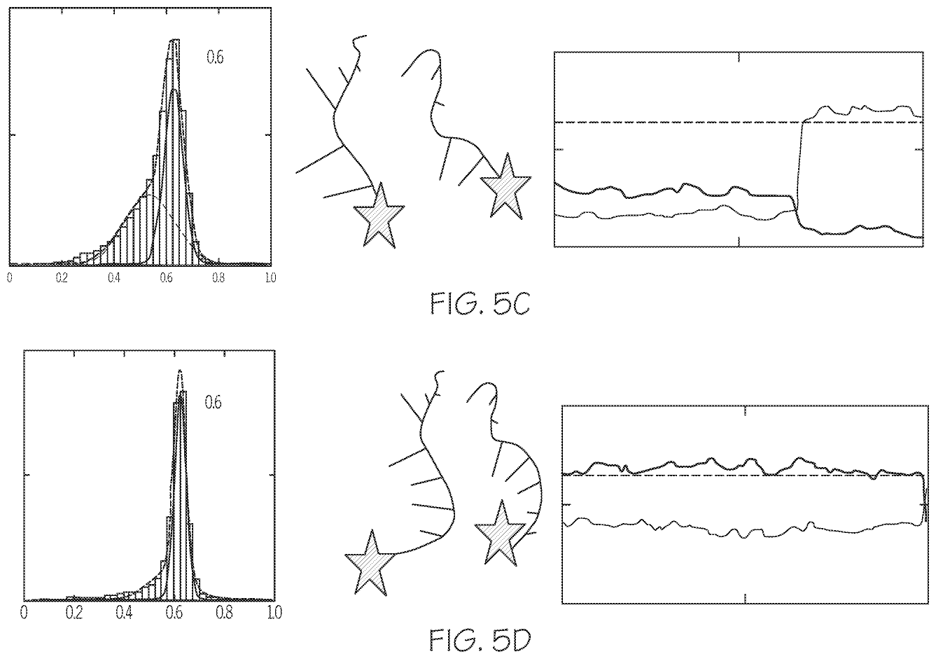

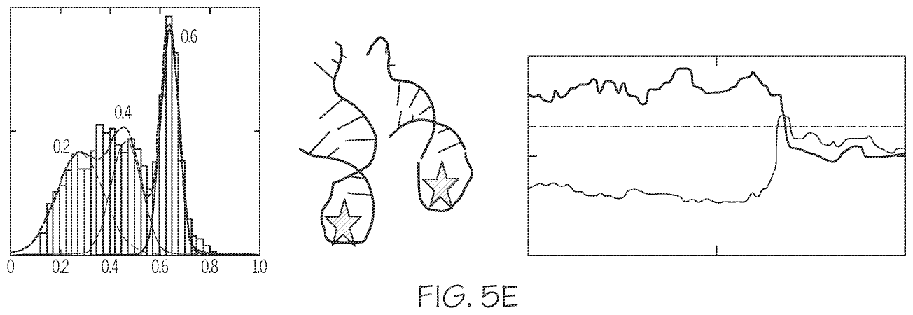

In Example 1, Applicant only used the short oligo “CACCACCA” from the RNase T1 digestion of the natural tRNA^{Lys}. Applicant has demonstrated other tRNA-like oligos that can be used as the ribozyme substrate, namely, a pentamer of DNA, a synthetic CACCACCA RNA, and a 20 nt minihelix. As shown by the high FRET efficiency histograms of FIGS. 5C-D, the binding and proximity for the minihelix is less efficient than DNA pentamer or the original octamers, but nevertheless a good substrate. The quality confirmation of these substrates was via Gel shifting, as illustrated in FIG. 5B.

### Example 2.3. Charging of Non-Natural Amino Acids Beyond Lysine

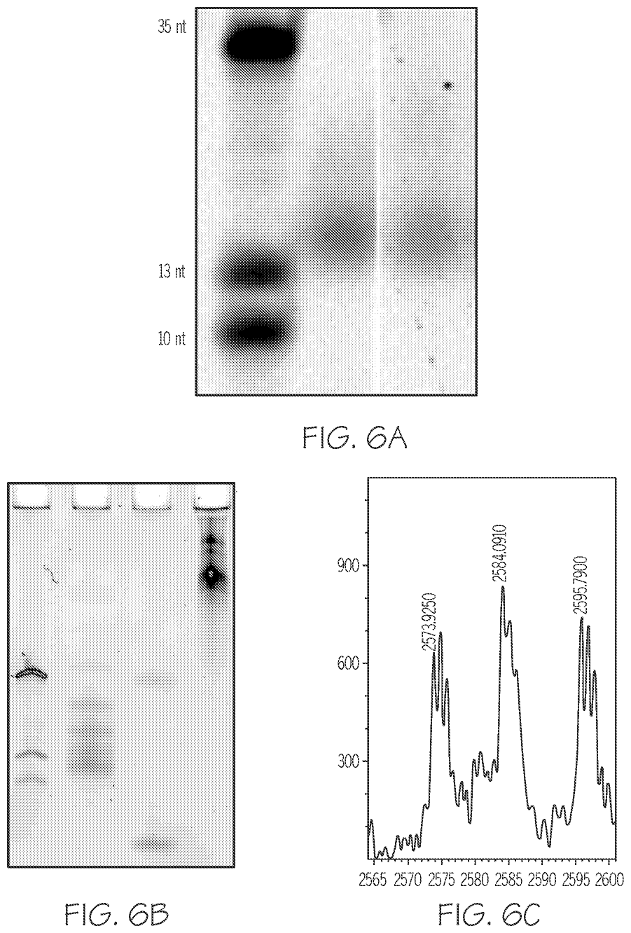

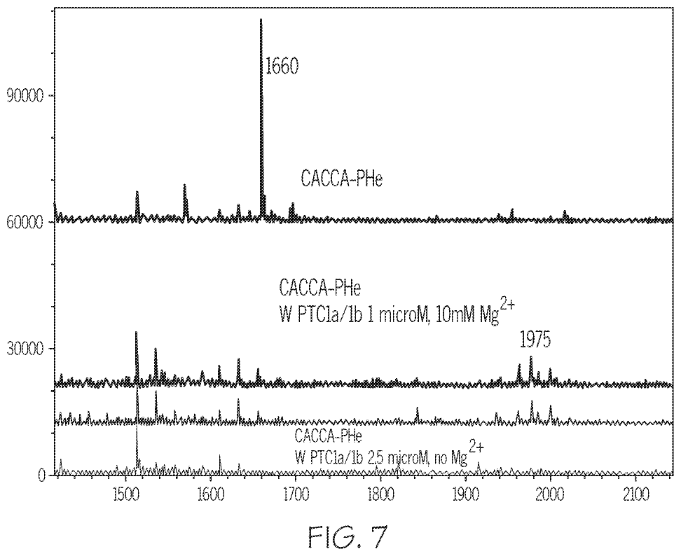

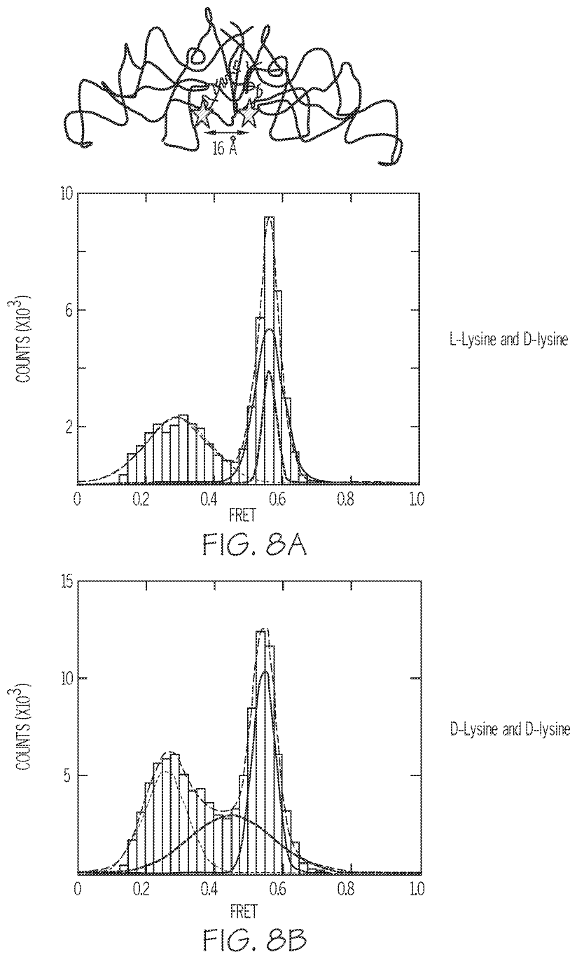

In Example 1, Applicant demonstrated peptide synthesis through the utilization of L-lysine. In this Example, Applicant demonstrates the successful charging of other non-natural amino acids, specifically, D-Cysteine and D-lysine on the tRNA like oligos. These two amino acids are chosen because their side residues can be fluorescently labeled and confirmed by fluorescence Gel shifting, as shown in FIGS. 6A-C. On the other hand, the formation of CCA (8 nt)-D-lysine is detected by high-resolution MassSpec.

### Example 2.4. Mass Spec Evidence to Show Peptide Ligation of Other Amino Acids

Applicant has demonstrated a tri-peptide formation from charged oligo CACCA-Phe (MW 1660), which is formed from RNaseT1 digestion of natural Phenylalanine-tRNA^{Phe}. As shown in FIG. 7, the mass peak at 1660 (brown trace) generated peaks around 1975 in the presence of PTC1a/1b (the patent ribozyme), with 10 mM Mg²⁺ (blue trace). Increasing the ribozyme concentration to 2.5 microM (pink trace) did not change the peptide formation but leaving out of Mg²⁺ abolished its formation (green trace). The calculated CACCA-(Phe)₃ is 1954, which is a −21 Dalton difference to the observed 1975 peak. This difference can be explained as the Na⁺ adduct.

### Example 2.5. Single Molecule FRET to Show Substrates Proximity

The function of non-natural amino acid charged oligos were demonstrated with single molecule FRET experiments. As shown in FIGS. 8A-8B, FRET-pair labeled Cy3-LK (L-Lysine)-CCA (8 nt) and Cy5-DK (D-Lysinee)-CCA (8 nt) generated high FRET efficiency, indicating that these non-natural amino acid charged oligos can function similar to the lysine-charged oligos, which has been demonstrated in Example 1.

Without further elaboration, it is believed that one skilled in the art can, using the description herein, utilize the present disclosure to its fullest extent. The embodiments described herein are to be construed as illustrative and not as constraining the remainder of the disclosure in any way whatsoever. While the embodiments have been shown and described, many variations and modifications thereof can be made by one skilled in the art without departing from the spirit and teachings of the invention. Accordingly, the scope of protection is not limited by the description set out above but is only limited by the claims, including all equivalents of the subject matter of the claims. The disclosures of all patents, patent applications, and publications cited herein are hereby incorporated herein by reference, to the extent that they provide procedural or other details consistent with and supplementary to those set forth herein.

What is claimed is:

1. A method of synthesizing a peptide, said method comprising:
associating at least one isolated ribosomal RNA (rRNA) fragment with a plurality of transfer RNA fragments linked to amino acids (tRNA fragments),
wherein the at least one rRNA fragment comprises at least one domain that associates with the tRNA fragments,
wherein the at least one rRNA fragment catalyzes the synthesis of the peptide through peptidyl transfer of the amino acids from the tRNA fragments,
wherein the peptidyl transfer occurs independently of messenger RNA (mRNA) templates, and
wherein the peptidyl transfer occurs independently of proteins.

2. The method of claim 1, wherein the amino acids are selected from the group consisting of canonical amino acids, non-canonical amino acids, non-natural amino acids, and combinations thereof.

3. The method of claim 1, wherein the formed peptide comprises from about 2-10 amino acids.

4. (canceled)

5. The method of claim 1, wherein the at least one rRNA fragment comprises a ribosomal peptidyl transferase center (PTC).

6-7. (canceled)

8. The method of claim 1, wherein the at least one rRNA fragment comprises a first rRNA fragment and a second rRNA fragment, and wherein the first rRNA fragment and the second rRNA fragment are in the form of a heterodimer.

9. The method of claim 8,
wherein the first rRNA fragment comprises AUGUCGGCUCGUCGCAUCCUGGGACUGAAGAAGGUCCCAAGGGUUGGGCUGUUC GCCCAUUAAAGCGGCACGCGAGCUGGGUUCAGAACGUCGUGAGACAGUUCGGUC (SEQ ID NO: 1); a derivative of SEQ ID NO: 1, wherein the derivative shares at least 70% sequence identity with SEQ ID NO: 1; or combinations thereof, and
wherein the second rRNA fragment comprises AGACCCCGUGGAGCUUUACUGCAGCCGAAGAAGGCGGGCAGUUUGACUGGGGCG GUC AA AAAAGUUACCCC GGGGA UAACAGGCUGAUCUCGUGAGGAG GUUUGGCACCUC (SEQ ID NO: 2); a derivative of SEQ ID NO: 2, wherein the derivative shares at least 70% sequence identity with SEQ ID NO: 2, or combinations thereof.

10-12. (canceled)

13. The method of claim 1, wherein the at least one rRNA fragment further comprises a dimerization agent to promote dimerization of the at least one rRNA fragment.

14. The method of claim 13, wherein the dimerization agent comprises MgCl₂.

15. The method of claim 1, wherein the peptidyl transfer occurs without proofreading.

16. The method of claim 1, wherein the tRNA fragments are in the form of short RNAs (sRNAs), wherein the sRNAs have lengths of less than about 50 nucleotides.

17. The method of claim 16, wherein the sRNAs have lengths of less than about 10 nucleotides.

18. The method of claim 11, wherein the tRNA fragments comprise one or more mini-helices.

19. The method of claim 1, further comprising a step of preparing the tRNA fragments, wherein the preparing is facilitated by a charging agent that facilitates the linking of amino acids to tRNA fragments, and wherein the charging agent is a Yadb protein or a derivative thereof.

20-37. (canceled)

38. A method of preparing transfer RNA fragments linked to amino acids (tRNA fragments), said method comprising:
Associating fragments of transfer RNA with amino acids in the presence of a charging agent,
wherein the charging agent facilitates the linking of amino acids to the transfer RNA fragments to form the tRNA fragments, and
wherein the charging agent is a Yadb protein or a derivative thereof.

39. The method of claim 38, wherein the amino acids are selected from the group consisting of canonical amino acids, non-canonical amino acids, non-natural amino acids, and combinations thereof.

40. The method of claim 38, wherein the tRNA fragments are in the form of short RNAs (sRNAs), wherein the sRNAs have lengths of less than about 50 nucleotides.

41. The method of claim 40, wherein the sRNAs have lengths of less than about 20 nucleotides.

42. The method of claim 38, wherein the tRNA fragments comprise one or more mini-helices.

43. The method of claim 38, wherein the Yadb protein comprises a derivative of the Yadb protein, wherein the derivative of the Yadb protein comprises mutations that optimize the binding of the Yadb protein to the tRNA fragments.

44. The method of claim 38, wherein the Yadb protein or its derivative is defined by SEQ ID NO: 3 or a sequence that shares at least 60% sequence identity with SEQ ID NO: 3


SEQUENCE LISTING (3 sequences)

SEQ ID NO: 1 - RNA, 108 nt, Artificial Sequence
  Synthetic RNA Oligonucleotide
augucggcucgucgcauccugggacugaagaaggucccaaggguugggcuguucgcccauuaaagcggcacgcgagcuggguucagaacgucgugagacaguucgguc

SEQ ID NO: 2 - RNA, 111 nt, Artificial Sequence
  Synthetic RNA Oligonucleotide
agaccccguggagcuuuacugcagccgaagaaggcgggcaguuugacuggggcggucaaaaaaguuaccccggggauaacaggcugaucucgugaggagguuuggcaccuc

SEQ ID NO: 3 - protein, 342 aa, Artificial Sequence
  Yadb Protein-E. coli
MTDTQYIGRFAPSPSGELHFGSLIAALGSYLQARARQGRWLVRIEDIDPPREVPGAAETILRQLEHYGLHWDGDVLWQSQRHDAYREALAWLHEQGLSYYCTCTRARIQSIGGIYDGHCRVLHHGPDNAAVRIRQQHPVTQFTDQLRGIIHADEKLAREDFIIHRRDGLFAYNLAVVVDDHFQGVTGDPRPVLIAALQFLGQQAEAHWQDFSVEQILQSAVKNWRLTAVPEIVRGADLIEPTVRQISLYQLFGWKVPDYIHLPLALNPQGAKLSKQNHAPALPKGDPRPVLIAALQFLGQQAEAHWQDFSVEQILQSAVKNWRLTAVPESAIVNSTFSNASC